(19) United States
(12) Patent Application Publication
(10) Pub. No.: US 2021/0285034 A1
(43) Pub. Date: Sep. 16, 2021

(54) FORMAMIDE FREE TARGET ENRICHMENT COMPOSITIONS FOR NEXT-GENERATION SEQUENCING APPLICATIONS

(71) Applicant: Roche Sequencing Solutions, Inc., Pleasanton, CA (US)

(72) Inventors: Jonathan S. Choi, Oakland, CA (US); Garima Kushwaha, San Francisco, CA (US); Cynthia Moehlenkamp, Verona, WI (US); Duylinh Nguyen, American Canyon, CA (US); Denise Raterman, Madison, WI (US)

(21) Appl. No.: 17/263,441

(22) PCT Filed: Jul. 26, 2019
(86) PCT No.: PCT/EP19/70217

§ 371 Date: Jan. 26, 2021

Related U.S. Application Data
(60) Provisional application No. 62/711,106, filed on Jul. 27, 2018.

(51) Int. Cl. C12Q 1/6832; C12Q 1/6806

(52) U.S. Cl. CPC C12Q 1/6832; C12Q 1/6806

(57) ABSTRACT

The invention is a novel composition of a nucleic acid hybridization solution used in a sequencing workflow.

### FIELD OF THE INVENTION

The invention relates to the field of nucleic acid analysis and more specifically, to nucleic acid hybridization within the nucleic acid sequencing workflow.

### BACKGROUND OF THE INVENTION

Nucleic acid hybridization experiments use formamide to facilitate denaturation of doubled stranded nucleic acid and minimize secondary structure formation by single nucleic acid strands. Formamide is also necessary to increase specificity of hybridization by destabilizing non-perfect (partially mismatched) nucleic acid duplexes and facilitating disruption of such duplexes during post-hybridization washes. Because formamide is toxic and is considered hazardous, its use is disfavored in laboratory products in wide clinical use. A non-toxic alternative to formamide is needed. A suitable replacement must have the properties of facilitating denaturation and increasing hybridization specificity. In addition, the presence of the formamide replacement may not interfere with any downstream applications such as nucleic acid sequencing. The present invention discloses formamide replacements suitable for next-generation nucleic acid sequencing applications.

### SUMMARY OF THE INVENTION

The invention is a sample preparation and sequencing workflow that includes a target enrichment step without the use of formamide. A replacement solvent selected from dimethyl sulfoxide (DMSO), sulfolane, ethylene carbonate, pyrrolidone or a primary amide is used.

In one embodiment, the invention is a method of enriching for a target nucleic acid in a solution of nucleic acids comprising the steps of: isolating nucleic acids in a sample solution; contacting the sample solution with a formamide-free hybridization solution comprising one or more single-stranded hybridization probe linked to a binding moiety and further comprising a solvent selected from dimethyl sulfoxide (DMSO), sulfolane, ethylene carbonate, pyrrolidone or a primary amide; incubating the sample under conditions facilitating formation of hybrids between the sample nucleic acids and the probes; isolating the hybrids by capturing the binding moiety. The pyrrolidone or amide has a structure selected from

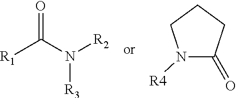

wherein R1 is H, methyl, propyl, or hydroxyethyl; R2 and R3 are independently of each other H or methyl; and R4 is H, propyl or isobutyl, e.g., the pyrrolidone or amide is selected from 2-pyrrolidone, N-methyl-pyrrolidone, N-hydroxyethyl pyrrolidone, acetamide, N-methylacetamide, N,N-dimethyl acetamide, propionamide, isobutyramide.

In one embodiment, the invention is a method of enriching for target nucleic acids to be sequenced by a single-molecule sequencing by synthesis, the method comprising the steps of: isolating nucleic acids in a sample solution; conjugating the nucleic acids to adaptors, wherein the adaptors comprise universal primer binding sites and sequencing primer binding sites; amplifying the adapted target nucleic acids with universal primers to form target amplicons; contacting the sample with a formamide-free hybridization solution comprising one or more single-stranded hybridization probes linked to a binding moiety and further comprising a solvent selected from dimethyl sulfoxide (DMSO), sulfolane, ethylene carbonate, pyrrolidone or a primary amide; incubating the sample under conditions facilitating formation of hybrids between the target amplicons and the probes; isolating the hybrids by capturing the binding moiety, releasing amplicons from the hybrids. In some embodiments, the pyrrolidone or amide has a structure selected from

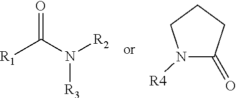

wherein R1 is H, methyl, propyl, or hydroxyethyl; R2 and R3 are independently of each other H or methyl; and R4 is H, propyl or isobutyl, e.g., the pyrrolidone or amide is selected from 2-pyrrolidone, N-methyl-pyrrolidone, N-hydroxyethyl pyrrolidone, acetamide, N-methylacetamide, N,N-dimethyl acetamide, propionamide, isobutyramide.

In one embodiment, the invention is a method of sequencing target nucleic acid comprising the steps of: isolating nucleic acids in a sample solution; conjugating the nucleic acids to adaptors, wherein the adaptors comprise universal primer binding sites and sequencing primer binding sites; amplifying the adapted target nucleic acids with universal primers to form target amplicons; contacting the sample with a formamide-free hybridization solution comprising one or more single-stranded hybridization probes linked to a binding moiety and further comprising and further comprising a solvent selected from dimethyl sulfoxide (DMSO), sulfolane, ethylene carbonate, pyrrolidone or a primary amide; incubating the sample under conditions facilitating formation of hybrids between the target amplicons and the probes; isolating the hybrids by capturing the binding moiety; releasing amplicons from the hybrids and sequencing the amplicons by extending the sequencing primer binding to the sequencing primer binding sites. In some embodiments, the pyrrolidone or amide has a structure selected from

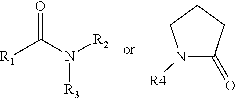

wherein R1 is H, methyl, propyl, or hydroxyethyl; R2 and R3 are independently of each other H or methyl; and R4 is H, propyl or isobutyl, e.g., the pyrrolidone or amide is selected from 2-pyrrolidone, N-methyl-pyrrolidone, N-hydroxyethyl pyrrolidone, acetamide, N-methylacetamide, N,N-dimethyl acetamide, propionamide, isobutyramide. In some embodiments, the sequencing is characterized by a performance characteristic equal to that of a method utilizing formamide-containing solution, wherein the characteristic is selected from read-on-target, deduplicated (deduped) depth, error rate, uniformity and GE recovery. For example, the characteristic is read-on-target, and the read-on-target is about 70% or greater. In another embodiment, the characteristic is deduplicated depth, and the deduplicated depth is 2500 or higher. In yet another embodiment, the characteristic is error rate, and the error rate is 0.04 or lower. In another embodiment the characteristic is uniformity, and the uniformity is about 2.5. In another embodiment, the characteristic is genome equivalent recovery, and the genome equivalent recovery is 0.25 or greater.

In one embodiment, the invention is a kit for enriching for target nucleic acids to be sequenced by a single-molecule sequencing by synthesis, the kit comprising reagents for isolating nucleic acids in a sample solution; conjugating the nucleic acids to adaptors, wherein the adaptors comprise universal primer binding sites and sequencing primer binding sites; amplifying the adapted target nucleic acids with universal primers to form target amplicons; hybridizing the amplified adapted target nucleic acids to one or more single-stranded hybridization probes linked to a binding moiety in a hybridization solution comprising a solvent selected from dimethyl sulfoxide (DMSO), sulfolane, ethylene carbonate, pyrrolidone or a primary amide. In some embodiments, the pyrrolidone or amide has a structure selected from

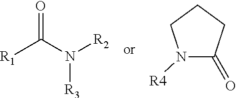

wherein R1 is H, methyl, propyl, or hydroxyethyl; R2 and R3 are independently of each other H or methyl; and R4 is H, propyl or isobutyl, e.g., the pyrrolidone or amide is selected from 2-pyrrolidone, N-methyl-pyrrolidone, N-hydroxyethyl pyrrolidone, acetamide, N-methylacetamide, N,N-dimethyl acetamide, propionamide, isobutyramide. In some embodiments, DMSO concentration in the hybridization buffer is selected from 15%, 18%, 20%, 23% 25%, 28%, 30% and 32%.

### BRIEF DESCRIPTION OF THE DRAWINGS

FIG. 1 illustrates performance of the novel hybridization buffers with 20% of formamide replacement in the sequencing workflow as measured by % of reads on target.

FIG. 2 illustrates performance of the novel hybridization buffers with 20% of formamide replacement in the sequencing workflow as measured by deduplicated depth.

FIG. 3 illustrates performance of the novel hybridization buffers with 20% of formamide replacement in the sequencing workflow as measured by uniformity.

FIG. 4 illustrates performance of the novel hybridization buffers with 20% of formamide replacement in the sequencing workflow as measured by error rate.

FIG. 5 illustrates performance of the novel hybridization buffers with 20% of formamide replacement in the sequencing workflow as measured by genome equivalent recovery.

FIG. 6 illustrates performance of the novel hybridization buffers with titration of formamide replacements in the sequencing workflow as measured by % of reads on target.

FIG. 7 illustrates performance of the novel hybridization buffers with titration of formamide replacements in the sequencing workflow as measured by deduplicated depth.

FIG. 8 illustrates performance of the novel hybridization buffers with titration of formamide replacements in the sequencing workflow as measured by uniformity.

FIG. 9 illustrates performance of the novel hybridization buffers with titration of formamide replacements in the sequencing workflow as measured by genome equivalent recovery.

FIG. 10 illustrates performance of the novel DMSO-containing hybridization buffers in the sequencing workflow as measured by % of reads on target.

FIG. 11 illustrates performance of the novel DMSO-containing hybridization buffers in the sequencing workflow as measured by deduplicated depth.

FIG. 12 illustrates performance of the novel DMSO-containing hybridization buffers in the sequencing workflow as measured by uniformity.

### DETAILED DESCRIPTION OF THE INVENTION

### Definitions

The following definitions aid in understanding of this disclosure.

The term “sample” refers to any composition containing or presumed to contain target nucleic acid. This includes a sample of tissue or fluid isolated from an individual for example, skin, plasma, serum, spinal fluid, lymph fluid, synovial fluid, urine, tears, blood cells, organs and tumors, and also to samples of in vitro cultures established from cells taken from an individual, including the formalin-fixed paraffin embedded tissues (FFPET) and nucleic acids isolated therefrom. A sample may also include cell-free material, such as cell-free blood fraction that contains cell-free DNA (cfDNA) or circulating tumor DNA (ctDNA).

The term “nucleic acid” refers to polymers of nucleotides (e.g., ribonucleotides and deoxyribonucleotides, both natural and non-natural) including DNA, RNA, and their subcategories, such as cDNA, mRNA, etc. A nucleic acid may be single-stranded or double-stranded and will generally contain 5′-3′ phosphodiester bonds, although in some cases, nucleotide analogs may have other linkages. Nucleic acids may include naturally occurring bases (adenosine, guanosine, cytosine, uracil and thymidine) as well as non-natural bases. Some examples of non-natural bases include those described in, e.g., Seela et al., (1999) Helv. Chim. Acta 82:1640. The non-natural bases may have a particular function, e.g., increasing the stability of the nucleic acid duplex, inhibiting nuclease digestion or blocking primer extension or strand polymerization.

The terms “polynucleotide” and “oligonucleotide” are used interchangeably. Polynucleotide is a single-stranded or a double-stranded nucleic acid. Oligonucleotide is a term sometimes used to describe a shorter polynucleotide. Oligonucleotides are prepared by any suitable method known in the art, for example, by a method involving direct chemical synthesis as described in Narang et al. (1979) Meth. Enzymol. 68:90-99; Brown et al. (1979) Meth. Enzymol. 68:109-151; Beaucage et al. (1981) Tetrahedron Lett. 22:1859-1862; Matteucci et al. (1981) J. Am. Chem. Soc. 103:3185-3191.

The term “hybridization” refers to the pairing of complementary nucleic acids to form a duplex (a double-stranded nucleic acid). Hybridization and the strength of hybridization (e.g., stability of the duplex) is affected by multiple factors including the degree of complementary between the nucleic acids, GC content of the nucleic acids and stringency of the hybridization and wash conditions involved.

The term “stringent conditions” or “high stringency conditions,” refers to hybridization conditions where only high-stability duplexes are formed. High stringency conditions typically include one or more of low salt and elevated temperature. For example, a traditional high stringency hybridization buffer at 42° C. may contain 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 mg/ml), 0.1% SDS, and 10% dextran sulfate.

The term “stringent wash” refers to post-hybridization wash with wash buffers containing successively lower concentrations of salts or higher concentrations of detergents and at increased temperatures. Stringent wash conditions may include temperatures in excess of about 42° C. Stringent wash buffer composition will typically contain less than about 0.1 M salt. For example, a traditional high stringency post-hybridization wash at 42° C. may contain 0.2×SSC (0.03 M NaCl, 0.003 M sodium citrate) and 50% formamide followed by a wash at 55° C. with 0.1×SSC.

The term “primer” refers to a single-stranded oligonucleotide which hybridizes with a sequence in the target nucleic acid (“primer binding site”) and is capable of acting as a point of initiation of synthesis along a complementary strand of nucleic acid under conditions suitable for such synthesis.

The term “adaptor” means a nucleotide sequence that may be added to another sequence so as to import additional properties to that sequence. An adaptor is typically an oligonucleotide that can be single- or double-stranded, or may have both a single-stranded portion and a double-stranded portion.

The term “ligation” refers to a condensation reaction joining two nucleic acid strands wherein a 5′-phosphate group of one molecule reacts with the 3′-hydroxyl group of another molecule. Ligation is typically an enzymatic reaction catalyzed by a ligase or a topoisomerase. Ligation may join two single strands to create one single-stranded molecule. Ligation may also join two strands each belonging to a double-stranded molecule thus joining two double-stranded molecules. Ligation may also join both strands of a double-stranded molecule to both strands of another double-stranded molecule thus joining two double-stranded molecules. Ligation may also join two ends of a strand within a double-stranded molecule thus repairing a nick in the double-stranded molecule.

The term “barcode” refers to a nucleic acid sequence that can be detected and identified. Barcodes can be incorporated into various nucleic acids. Barcodes are sufficiently long e.g., 2, 5, 20 nucleotides, so that in a sample, the nucleic acids incorporating the barcodes can be distinguished or grouped according to the barcodes.

The term “multiplex identifier” or “MID” refers to a barcode that identifies a source of a target nucleic acids (e.g., a sample from which the nucleic acid is derived). All or substantially all the target nucleic acids from the same sample will share the same MID. Target nucleic acids from different sources or samples can be mixed and sequenced simultaneously. Using the MIDs the sequence reads can be assigned to individual samples from which the target nucleic acids originated.

The term “unique molecular identifier” or “UID” refers to a barcode that identifies a nucleic acid to which it is attached. All or substantially all the target nucleic acids from the same sample will have different UIDs. All or substantially all of the progeny (e.g., amplicons) derived from the same original target nucleic acid will share the same UID.

The term “universal primer” and “universal priming binding site” or “universal priming site” refer to a primer and primer binding site present in (typically, through in vitro addition to) different target nucleic acids. The universal priming site is added to the plurality of target nucleic acids using adaptors or using target-specific (non-universal) primers having the universal priming site in the 5′-portion. The universal primer can bind to and direct primer extension from the universal priming site.

The terms “target sequence”, “target nucleic acid” or “target” refer to a portion of the nucleic acid sequence in the sample which is to be detected or analyzed. The term target includes all variants of the target sequence, e.g., one or more mutant variants and the wild type variant.

The term “amplification” refers to a process of making additional copies of the target nucleic acid. Amplification can have more than one cycle, e.g., multiple cycles of exponential amplification. Amplification may also have only one cycle (making a single copy of the target nucleic acid). The copy may have additional sequences, e.g., those present in the primers used for amplification.

The term “sequencing” refers to any method of determining the sequence of nucleotides in the target nucleic acid. “Next generation sequencing,” “massively parallel sequencing” and “massively parallel single molecule sequencing” are used interchangeably to refer to sequencing in-parallel of an entire population of isolated individual nucleic acids (or nucleic acid amplicons).

The present invention comprises a nucleic acid sequencing workflow wherein the sequencing is next-generation sequencing also referred to as massively parallel singe-molecule sequencing. In some embodiments, the workflow comprises the steps of nucleic acid isolation, sequencing library preparation, target enrichment and sequencing. The step of target enrichment is novel and comprises the use of novel formamide-free compositions and methods that involve the use of formamide alternatives that are compatible with (do not inhibit or interfere with) the sequencing process. A formamide alternative is selected from solvents such as dimethyl sulfoxide (DMSO), sulfolane, ethylene carbonate, pyrrolidone or a primary amide. The pyrrolidone or a primary amide has a structure selected from

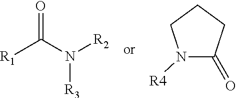

wherein R1 is H, methyl, propyl, or hydroxyethyl; R2 and R3 are independently of each other H or methyl; and R4 is H, propyl or isobutyl. In some embodiments, the pyrrolidone or amide is selected from 2-pyrrolidone, N-methyl-pyrrolidone, N-hydroxyethyl pyrrolidone, acetamide, N-methylacetamide, N,N-dimethyl acetamide, propionamide, isobutyramide.

The present invention comprises detecting a target nucleic acid in a sample. In some embodiments, the sample is derived from a subject or a patient. In some embodiments the sample may comprise a fragment of a solid tissue or a solid tumor derived from the subject or the patient, e.g., by biopsy. The sample may also comprise body fluids (e.g., urine, sputum, serum, plasma or lymph, saliva, sputum, sweat, tear, cerebrospinal fluid, amniotic fluid, synovial fluid, pericardial fluid, peritoneal fluid, pleural fluid, cystic fluid, bile, gastric fluid, intestinal fluid, and/or fecal samples), The sample may comprise whole blood or blood fractions where tumor cells may be present. In some embodiments, the sample, especially a liquid sample may comprise cell-free material such as cell-free DNA or RNA including cell-free tumor DNA or tumor RNA. In some embodiments, the sample is a cell-free sample, e.g., cell-free blood-derived sample where cell-free tumor DNA or tumor RNA are present. In other embodiments, the sample is a cultured sample, e.g., a culture or culture supernatant containing or suspected to contain an infectious agent or nucleic acids derived from the infectious agent. In some embodiments, the infectious agent is a bacterium, a protozoan, a virus or a mycoplasma.

A target nucleic acid is the nucleic acid of interest that may be present in the sample. In some embodiments, the target nucleic acid is a gene or a gene fragment. In other embodiments, the target nucleic acid contains a genetic variant, e.g., a polymorphism, including a single nucleotide polymorphism or variant (SNP of SNV), or a genetic rearrangement resulting e.g., in a gene fusion. In some embodiments, the target nucleic acid comprises a biomarker. In other embodiments, the target nucleic acid is characteristic of a particular organism, e.g., aids in identification of the pathogenic organism or a characteristic of the pathogenic organism, e.g., drug sensitivity or drug resistance. In yet other embodiments, the target nucleic acid is characteristic of a human subject, e.g., the HLA or KIR sequence defining the subject's unique HLA or KIR genotype. In yet other embodiments, all the sequences in the sample are target nucleic acids e.g., in shotgun genomic sequencing.

In some embodiments, the sequencing workflow comprises a step of amplifying the target nucleic acid. The amplification may be by polymerase chain reaction (PCR) or any other method that utilizes oligonucleotide primers. Various PCR conditions are described in PCR Strategies (M. A. Innis, D. H. Gelfand, and J. J. Sninsky eds., 1995, Academic Press, San Diego, Calif.) at Chapter 14; PCR Protocols: A Guide to Methods and Applications (M. A. Innis, D. H. Gelfand, J. J. Sninsky, and T. J. White eds., Academic Press, N Y, 1990).

The amplification may utilize bipartite amplification primers comprising a target-specific sequence and artificial sequence needed for subsequent steps (adaptor sequence). In some embodiments, a defined target or group of target nucleic acids is being interrogated. In such embodiments, target specific amplification primers may be used. A primer may have a bipartite structure composed of a target-specific sequence in the 3′-portion and an adaptor sequence in the 5′-portion. Typically, the target-specific primers are used as a pair of distinct oligonucleotides, e.g., a forward and a reverse primer. The 5′-portion may also comprise a universal primer binding site to enable amplification with universal primers. The 5′-portion may also comprise a sequencing primer binding site to enable sequencing with sequencing primers, e.g., platform-specific sequencing primers.

In some embodiments, the sequencing workflow comprises a step of library preparation. The library preparation commences with adaptor ligation. Adaptors are introduced into the target nucleic acids either by primer extension, by PCR amplification or by ligation. In some embodiments, the primer extension is a single round. In other embodiments, the primer extension goes through multiple cycles, e.g., PCR amplification (as set forth in the preceding section). The resulting target nucleic acid comprises a target sequence flanked by the adaptor sequences. In some embodiments, adaptors contain primer binding sites for downstream steps, e.g., amplification primer binding sites and sequencing primer binding sites.

Adaptor ligation can be a blunt-end ligation or a more efficient cohesive-end ligation. In some embodiments, target nucleic acid or the adaptors may be rendered blunt-ended by strand-filling, i.e., extending a 3′-terminus by a DNA polymerase to eliminate a 5′-overhang or digesting the 3′-overhang with a 3′-5′-ecxonuclease activity. In some embodiments, the blunt-ended adaptors and target nucleic acid may be rendered cohesive by addition of a single nucleotide to the 3′-end of the adaptor and a single complementary nucleotide to the 3′-ends of the target nucleic acid, e.g., by a DNA polymerase or a terminal transferase. In yet other embodiments, the adaptors and the target nucleic acid may acquire cohesive ends (overhangs) by digestion with restriction endonucleases. The latter option is more advantageous for known target sequences that are known to contain the restriction enzyme recognition site. In each of the above embodiments, the adaptor molecule may acquire the desired ends (blunt, single-base extension or multi-base overhang) by design of the synthetic adaptor oligonucleotides further described below. In some embodiments, the adaptor molecules are in vitro synthesized artificial sequences. In other embodiments, the adaptor molecules are in vitro synthesized naturally-occurring sequences known to possess the desired secondary structure. In yet other embodiments, the adaptor molecules are isolated naturally occurring molecules or isolated non naturally-occurring molecules.

In some embodiments of the sequencing workflow, the adaptors comprise one or more barcodes. A barcode can be a multiplex sample ID (MID) used to identify the source of the sample where samples are mixed (multiplexed). The barcode may also serve as a unique molecular ID (UID) used to identify each original molecule and its progeny. The barcode may also be a combination of a UID and an MID. In some embodiments, a single barcode is used as both UID and MID. In some embodiments, each barcode comprises a predefined sequence. In other embodiments, the barcode comprises a random sequence. Barcodes can be 1-20 nucleotides long.

The method of the invention comprises a novel target enrichment step. The enrichment may be by capturing the target sequences via hybridization to one or more target-specific probes. In some embodiments, the hybridization probes comprise one or more sequences that target a plurality of exons, introns, or regulatory sequences from a plurality of genetic loci, or complete sequences of at least one single genetic locus, said locus having a size of between about 100 kb and about 1 Mb.

In some embodiments, the novel hybridizations solutions of the sequencing workflow comprise solvents described in Matthiesen, S., et al., (2012) Fast and Non-Toxic In Situ Hybridization without Blocking of Repetitive Sequences PLoS ONE, 7: e40675 or U.S. Pat. No. 9,303,287. In some embodiments, a formamide replacement is a polar aprotic solvent selected based on its Hansen Solubility Parameters as described in Hansen, Charles (2007). Hansen Solubility Parameters: A user's handbook, Second Edition. Boca Raton, Fla.: CRC Press. The parameters measure certain energy characteristics of the solvent and are expressed in MPa^{0.5}. Specifically, the solvent is chosen if its D parameter was between 17.7 to 22.0 MPa^{0.5}, the P parameter is between 13 to 23 MPa^{0.5} and the H parameter is between 3 to 13 MPa^{0.5}.

In some embodiments, the novel hybridizations solutions of the sequencing workflow comprise solvents described in Chakrabarti, R. et al. (2001) The enhancement of PCR amplification by low molecular weight amides. N.A.R. 29:2377. In some embodiments, the formamide replacement is a solvent selected from dimethyl sulfoxide (DMSO), sulfolane, ethylene carbonate, pyrrolidone and a primary amide. In some embodiments, the pyrrolidone or amide has a structure selected from

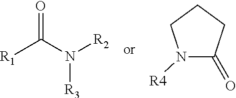

wherein R1 is H, methyl, propyl, or hydroxyethyl; R2 and R3 are independently of each other H or methyl; and R4 is H, propyl or isobutyl. In some embodiments, the solvent is selected from 2-pyrrolidone, N-methyl-pyrrolidone, N-hydroxyethyl pyrrolidone, acetamide, N-methylacetamide, N,N-dimethyl acetamide, propionamide, isobutyramide.

The nucleic acids in the sample are single stranded or are denatured and contacted with single-stranded target-specific probes in a novel hybridization buffer containing the formamide replacement according to the invention. The probes may comprise a ligand for an affinity capture moiety so that after hybridization complexes are formed, the complexes are captured by providing the affinity capture moiety. In some embodiments, the affinity capture moiety is avidin or streptavidin and the ligand is biotin. Other examples of the ligand-capture moiety include digoxigenin/anti-digoxigenin and 6HIS/nickel. In some embodiments, the capture moiety is bound to solid support. The solid support may comprise suspended particles such as superparamagnetic spherical polymer particles such as DYNABEADS™ magnetic beads or magnetic glass particles.

In embodiments of the present invention, a sample containing denatured (e.g., single-stranded) nucleic acid molecules is exposed to the formamide-free hybridization buffer containing one or more oligonucleotide probes.

The probes typically target one or more genomic loci by the use of one or more capture probes per locus. In some embodiments the probes target a panel of disease related genes, such as a panel of cancer associated genes and tumor associated-loci comprising. In some embodiments, the panels include AVENIO ctDNA panels selected from the Targeted panel for tumor profiling, Expanded panel for expanded tumor profiling and Surveillance panel for longitudinal tumor burden monitoring (Roche Sequencing Solutions, Pleasanton, Cal). The probes may be present in solution or may be bound to solid support such as beads or microarray. The probes hybridize to (i.e., capture) the target nucleic acid sequences. Subsequent post-hybridization wash separates non-hybridized nucleic acids, e.g., excess probes and non-hybridizing regions of the genome or any other non-target sample nucleic acids from the hybridized target sequences. In some embodiments, hybridization washes are under stringent conditions including one or both low salt and elevated temperatures. In some embodiments, the washes are performed at 47° C. or room temperature in standard and stringent wash buffers.

In some embodiments, the present invention comprises detecting target nucleic acids in a sample by nucleic acid sequencing. Multiple nucleic acids, including all the nucleic acids captured according to the method of the invention may be sequenced.

In some embodiments, sequencing is by high-throughput single molecule sequencing by synthesis methods such as Illumina platforms including HiSeq, MiSeq and NextSeg (Illumina, San Diego, Calif.). Other examples of sequencing methods and platforms include sequencing by synthesis Helicos Biosciences (Cambridge, Mass.), sequencing-by-ligation (e.g., SOLiD™) and ion semiconductor sequencing (e.g., Ion Torrent™) both from Thermo Fisher Scientific, the Pacific BioSciences platform utilizing the SMRT technology (Pacific Biosciences, Menlo Park, Calif.) or a platform utilizing nanopore technology such as those manufactured by Oxford Nanopore Technologies (Oxford, UK) or Roche Sequencing Solutions (Santa Clara, Calif.) and any other presently existing or future DNA sequencing technology that does or does not involve sequencing by synthesis. The sequencing step may utilize platform-specific sequencing primers. Binding sites for these primers may be introduced in the method of the invention as described herein, i.e., by being a part of adaptors or amplification primers.

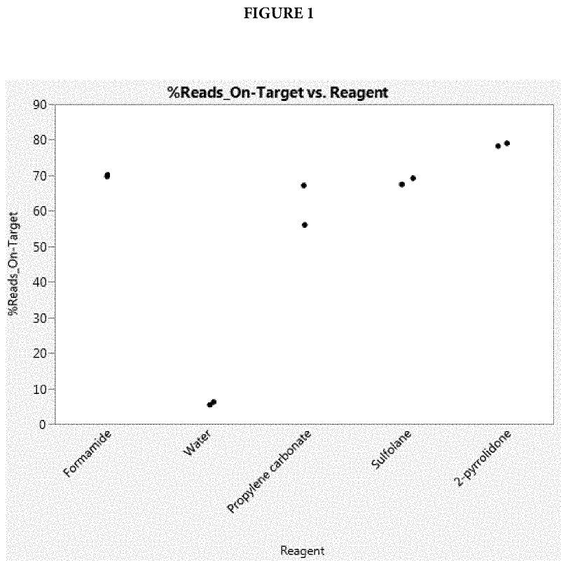

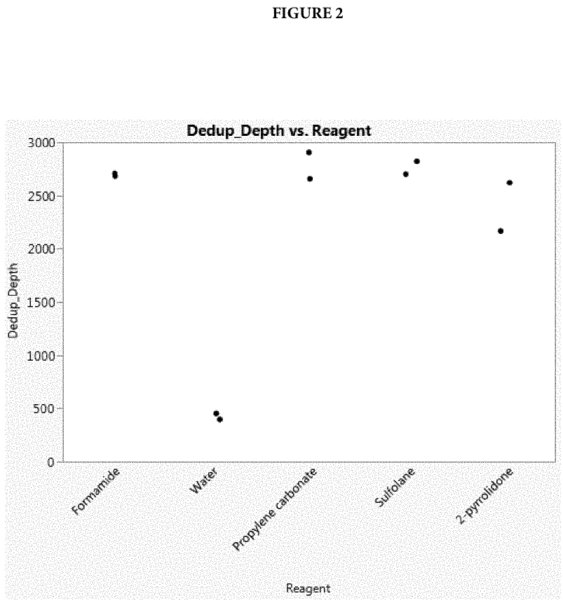

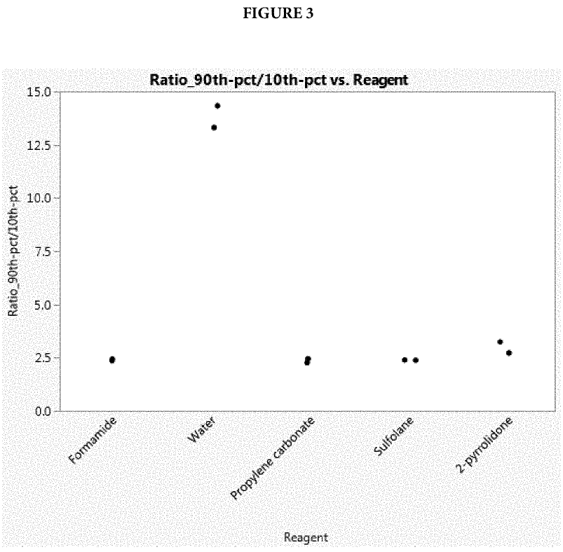

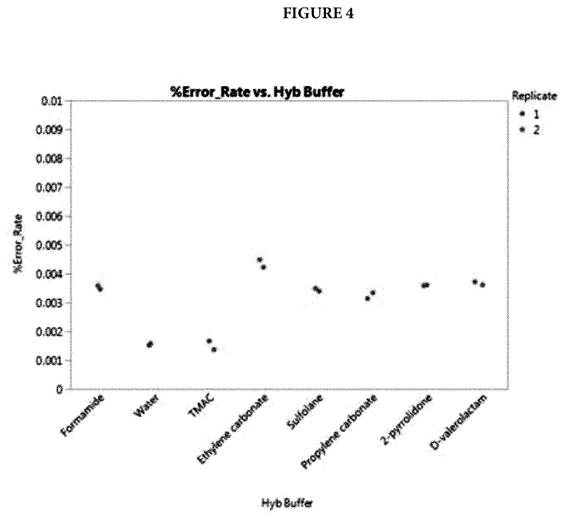

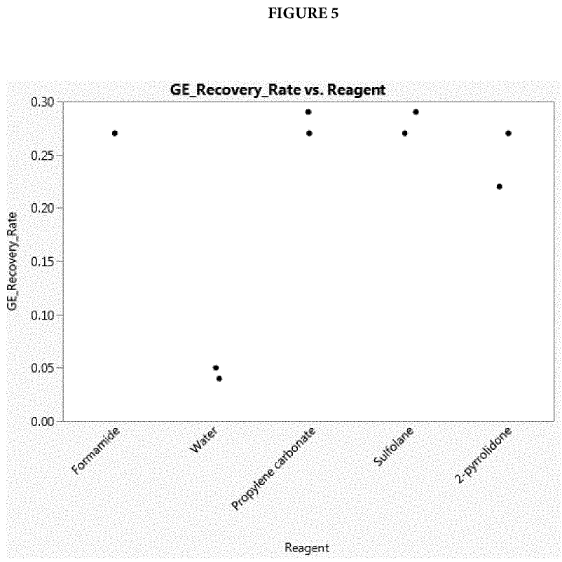

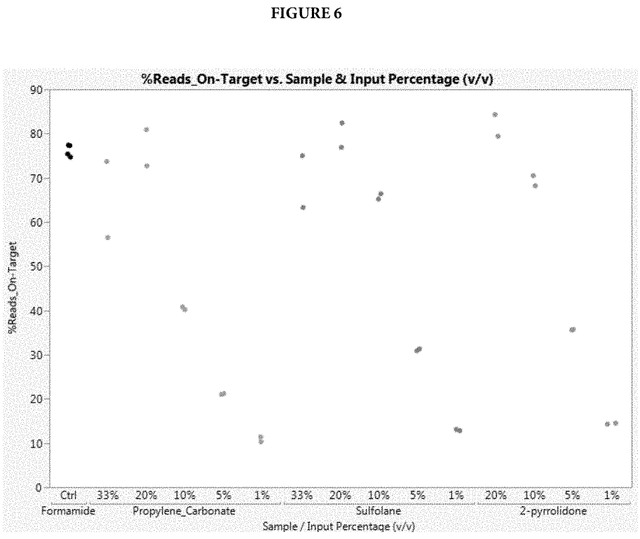

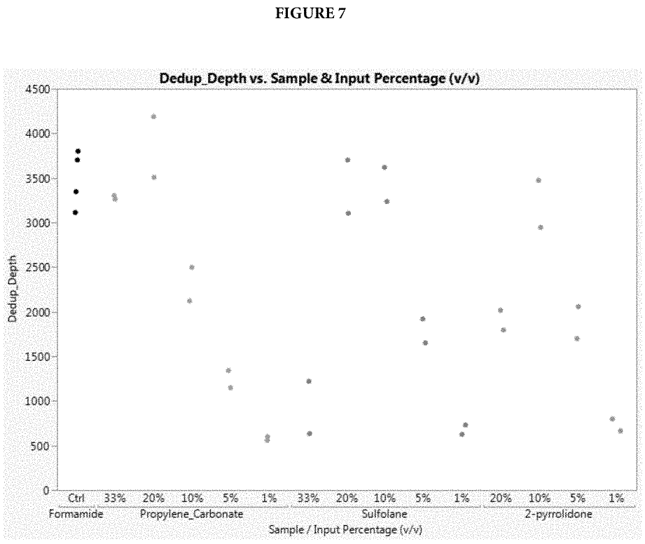

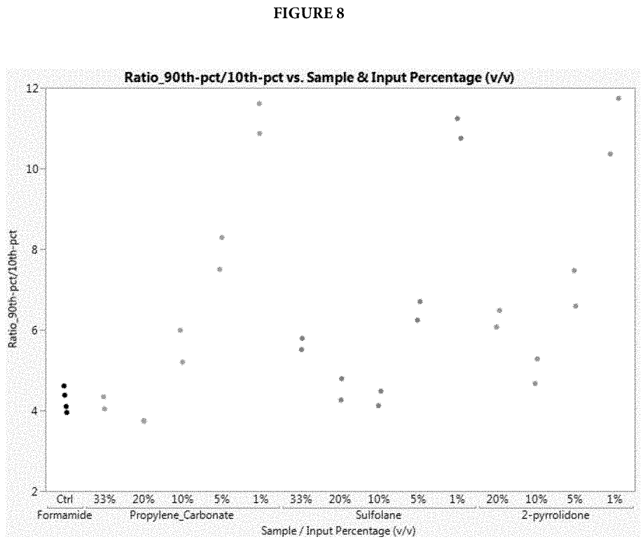

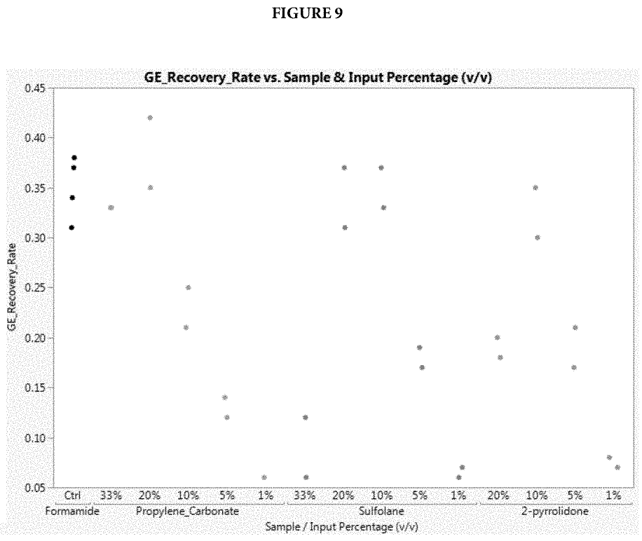

In some embodiments, the method utilizing novel formamide-free solutions is characterized by a sequencing-related performance characteristic. The characteristic is selected from read-on-target, deduplicated (deduped) depth, error rate, uniformity and GE recovery. As illustrated by FIGS. 1, 2, 3 and 4, when the formamide replacement is present at 20% in the hybridization solution, the characteristics are similar to or superior than those of a formamide containing hybridization solutions. In one aspect, the read-on-target characteristic is determined as percentage of mapped reads with any part of the read mapping to the target regions (defined by the panel). As shown on FIG. 1, read-on-target is about 70% or greater for both formamide-containing and novel formamide-free hybridization buffers. In one aspect, the deduplicated (deduped) depth characteristic is determined as average depth of on-target reads measured after deduplication. As shown on FIG. 2, the deduped depth is at or greater than 2500 for both formamide-containing and novel formamide-free hybridization buffers. In one aspect, the uniformity characteristic is determined as a 90th/10th ratio, or the ratio of 90th percentile base coverage to 10th percentile base coverage. As shown on FIG. 3, the uniformity for both formamide-containing and novel formamide-free hybridization buffers is 2.5 or greater. In one aspect, the error rate characteristic is determined as percentage of all bases within all reads that have non-reference alleles, wherein the calculation is restricted to 1) positions with a total read depth of at least 200 (after barcode deduplication), and 2) the non-reference bases have an allele fraction of at most 5%. As shown on FIG. 4, the error rate for both formamide-containing and novel formamide-free hybridization buffers is at or below 0.005%. In one aspect, the GE (genome equivalent) recovery characteristic is determined as the number of genome equivalents recovered. As shown on FIG. 5, the GE (genome equivalent) recovery for both formamide-containing and novel formamide-free hybridization buffers is between 0.2 and 0.3. As illustrated by FIGS. 6, 7, 8 and 9, when the amount of formamide replacement is titrated in the hybridization solution, an optimum can be found where the performance characteristics are similar to or superior than those of a formamide containing hybridization solutions.

In some embodiments, the invention is a kit for performing the target enrichment and sequencing method of the invention. The kit comprises a formamide-free hybridization solution comprising a solvent selected from sulfolane, ethylene carbonate, pyrrolidone or a primary amide, and one or more of the following: adaptors, universal amplification primers, enzymes, including a DNA ligase (T4 DNA ligase, Taq DNA ligase, or E. coli DNA ligase), a polynucleotide kinase and a DNA polymerase, such as an amplification polymerase or a sequencing polymerase. In some embodiments, the kit also includes an exonuclease with a 5′-3′-activity such as a T5 exonuclease.

### EXAMPLES

### Example 1. Sequencing Workflow with Formamide Replacements Propylene Carbonate, Sulfolane, and 2-Pyrrolidone

Briefly, in this example, the sequencing workflow was performed according to the AVENIO ctDNA assay (Roche Sequencing Solutions, Pleasanton, Calif.), except in the hybridization step of the target capture protocol, formamide was replaced with one of the aprotic solvents selected from with propylene carbonate, sulfolane, and 2-pyrrolidone. Captured libraries were sequenced on the Illumina NextSeq 500 (Ilumina, San Diego, Calif.) Sequencing results for libraries captured in the presence of formamide replacements demonstrated comparable results to the formamide control and substantially better than the water control. The workflow contained the steps of DNA fragmentation, library preparation, including adaptor ligation and PCR amplification, target enrichment, post-hybridization clean-up and sequencing.

The DNA fragmentation step was performed using the KAPA Frag Kit (Kapa Biosystems, Wortham, Mass.) according to the manufacturer's recommendations. Briefly, 100 ng of NA12878 genomic DNA was enzymatically fragmented at 37° C. using the (KR1141) and purified using affinity chromatography. The DNA was quantified using Qubit quality of fragmentation and was assessed with the High Sensitivity Bioanalyzer Kit.

The library preparation step utilized 30 ng of fragmented NA12878. 10 μl of universal adapter solution from the AVENIO kit was added to each sample and ligated for 16-18 hours at 16° C. The ligated DNA was purified using affinity chromatography and used in PCR amplification.

The PCR step utilized barcode-specific universal primers and the following temperature profile:

| Stage | Temperature | Duration | Cycles |
|---|---|---|---|
| Initial Denaturing | 98° C. | 45 seconds | 1 |
| Denaturing | 98° C. | 15 seconds | 8 |
| Annealing | 60° C. | 30 seconds | |
| Extension | 72° C. | 30 seconds | |
| Final extension | 72° C. | 60 seconds | 1 |
| Hold | 4° C. | ∞ | 1 |

The amplified ligated DNA was purified using affinity chromatography and used in the target enrichment step. Prior to that step, the library was quantified using Qubit and the size of library fragments was assessed with the High Sensitivity Bioanalyzer Kit.

The target enrichment step utilized 30 μl of adapter ligated sample. The reaction included Hybridization Supplement, and Enhancing Oligo from the AVENIO kit and was carried out according to the manufacturer's protocol except the hybridization solution included one of the following:

| Reagent | Final concentration |
|---|---|
| Formamide (control) | 20% |
| Propylene Carbonate | 20% |
| Sulfolane | 20% |
| 2-pyrrolidone | 20% |
| Water (control) | n/a |

The sample was subjected to the following temperature profile:

| | |
|---|---|
| 95° C. | 10 minutes |
| 47° C. | ∞ |

The hybridization wash step utilized the standard and stringent AVENIO hybridization wash buffers at 47° C. and room temperature. The hybridized nucleic acids were captured on streptavidin beads and used in the PCR amplification step.

The PCR amplification step utilized 20 μL of DNA bound to streptavidin beads. After adding the PCR reaction mixture, the reactions were subjected to the following temperature profile:

| Stage | Temperature | Duration | Cycles |
|---|---|---|---|
| Initial | 98° C. | 45 seconds | 1 |
| Denaturing | | | |
| Denaturing | 98° C. | 15 seconds | 15 |
| Annealing | 60° C. | 30 seconds | |
| Extension | 72° C. | 30 seconds | |
| Final extension | 72° C. | 60 seconds | 1 |
| Hold | 4° C. | ∞ | 1 |

PCR products were purified using affinity chromatography. The amount and quality of the DNA was assessed using Qubit and the High Sensitivity Bioanalyzer Kit. The PCR products were then sequenced on NextSeq 500/550 according to the manufacturer's protocols.

Results are shown in FIGS. 1, 2, 3, 4 and 5.

### Example 2. Sequencing Workflow with Titrated Formamide Replacements Propylene Carbonate, Sulfolane, and 2-Pyrrolidone

In this example, the sequencing workflow was performed as described in Example 1, except the following formamide replacements in the following concentrations were used:

| Reagent | Volume % |
|---|---|
| Formamide (control) | 20% |
| | 15% |
| | 10% |
| | 5% |
| Propylene Carbonate | 20% |
| | 15% |
| | 10% |
| | 5% |
| Sulfolane | 20% |
| | 15% |
| | 10% |
| | 5% |
| 2-pyrrolidone | 20% |
| | 15% |
| | 10% |
| | 5% |

Results are shown in FIGS. 6, 7, 8 and 9.

### Example 3. Sequencing Workflow with Formamide Replacement DMSO

Briefly, in this example, the sequencing workflow was performed according to the AVENIO Tumor Tissue DNA assay (Roche Sequencing Solutions, Pleasanton, Calif.), except in the hybridization step of the target capture protocol, formamide was replaced with DMSO. Captured libraries were sequenced on the Illumina NextSeq 500 (Ilumina, San Diego, Calif.) Sequencing results for libraries captured in the presence of DMSO demonstrated comparable results to the formamide control and substantially better than the water control. The workflow contained the steps of DNA polishing, DNA fragmentation, library preparation, including adaptor ligation and PCR amplification, target enrichment, post-hybridization clean-up, post-hybridization amplification and sequencing.

The DNA polishing step and utilized the DNA polishing enzyme included in the AVENIO Tumor Tissue DNA kit according to the manufacturer's instructions. The DNA fragmentation step was performed as described in Example 1. The library preparation step including A-tailing and adaptor ligation was performed essentially as described in Example 1 utilized 20 ng of genomic DNA from a cell line HD789 (Horizon Discovery). Ligation of universal adapters from the AVENIO kit was carried out for 16-18 hours at 16° C. The ligated DNA was purified using affinity chromatography and used in PCR amplification. The amplification utilized the temperature profile listed in Example 1.

The amplified ligated DNA was purified using affinity chromatography and used in the target enrichment step. Prior to that step, the library was quantified using Qubit and the size of library fragments was assessed with the High Sensitivity Bioanalyzer Kit.

The target enrichment step utilized 30 μl of adapter ligated sample. The reaction included Hybridization Supplement, clean-up beads and Enhancing Oligo from the AVENIO kit and was carried out according to the manufacturer's protocol except the hybridization solution included either 20% formamide (control) or DMSO at the following concentrations: 15%, 18%, 20%, 23% 25%, 28%, 30%, 32%, 38% and 40%.

The sample was subjected to the following temperature profile:

| | |
|---|---|
| 95° C. | 10 minutes |
| 55° C. | ∞ |

The hybridization wash step utilized the standard and stringent AVENIO hybridization wash buffers at 55° C. and room temperature. The hybridized nucleic acids were captured on streptavidin beads and used in the PCR amplification step. Post-capture PCR amplification was performed as described in Example 1.

PCR products were purified using affinity chromatography. The amount and quality of the DNA was assessed using Qubit and the High Sensitivity Bioanalyzer Kit. The PCR products were then sequenced on NextSeq 500/550 according to the manufacturer's protocols.

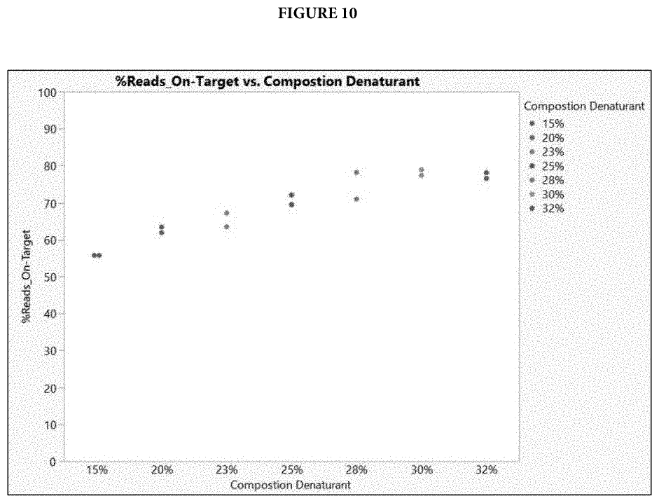

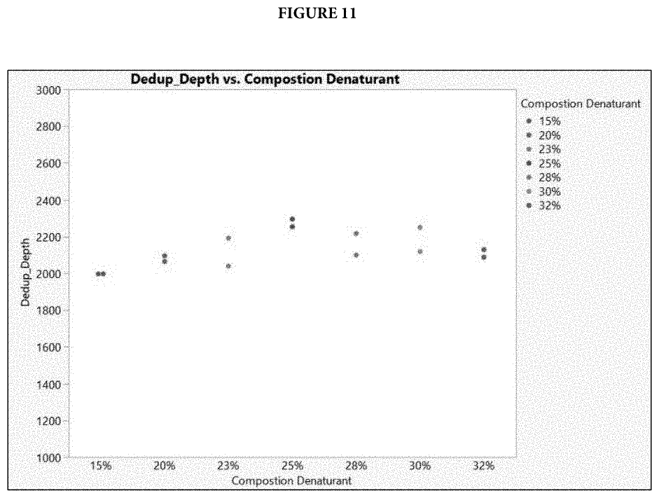

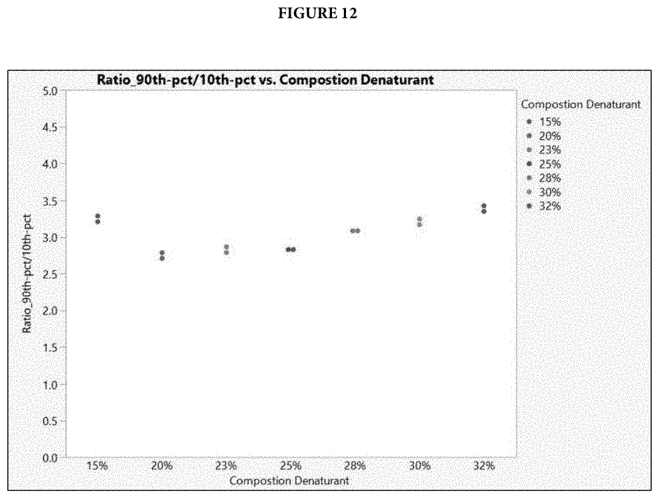

Results for DMSO concentrations: 15%, 18%, 20%, 23% 25%, 28%, 30%, 32%, 35%, 38% and 40% are shown in FIGS. 10, 11 and 12 (except sequence data could not be retrieved from conditions with 35%, 38%, and 40% DMSO).

1-3. (canceled)

4. A method of enriching for target nucleic acids to be sequenced by a single-molecule sequencing by synthesis, the method comprising the steps of:
a. isolating nucleic acids in a sample solution;
b. conjugating the nucleic acids to adaptors, wherein the adaptors comprise universal primer binding sites and sequencing primer binding sites;
c. amplifying the adapted target nucleic acids with universal primers to form target amplicons;
d. contacting the sample with a formamide-free hybridization solution comprising one or more single-stranded hybridization probes linked to a binding moiety and further comprising a solvent selected from dimethyl sulfoxide (DMSO), sulfolane, ethylene carbonate, pyrrolidone or a primary amide;
e. incubating the sample under conditions facilitating formation of hybrids between the target amplicons and the probes;
f. isolating the hybrids by capturing the binding moiety.
g. releasing amplicons from the hybrids.

5. The method of claim 4, wherein the pyrrolidone or amide has a structure selected from 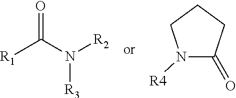
wherein R1 is H, methyl, propyl, or hydroxyethyl; R2 and R3 are independently of each other H or methyl; and R4 is H, propyl or isobutyl.

6. The method of claim 5, wherein the pyrrolidone is selected from 2-pyrrolidone, N-methyl-pyrrolidone, N-hydroxyethyl pyrrolidone, and the amide is selected from acetamide, N-methylacetamide, N,N-dimethyl acetamide, propionamide, isobutyramide.

7. A method of sequencing target nucleic acid comprising the steps of:
a. isolating nucleic acids in a sample solution;
b. conjugating the nucleic acids to adaptors, wherein the adaptors comprise universal primer binding sites and sequencing primer binding sites;
c. amplifying the adapted target nucleic acids with universal primers to form target amplicons;
d. contacting the sample with a formamide-free hybridization solution comprising one or more single-stranded hybridization probes linked to a binding moiety and further comprising and further comprising a solvent selected from dimethyl sulfoxide (DMSO), sulfolane, ethylene carbonate, pyrrolidone or a primary amide;
e. incubating the sample under conditions facilitating formation of hybrids between the target amplicons and the probes;
f. isolating the hybrids by capturing the binding moiety;
g. releasing amplicons from the hybrids and sequencing the amplicons by extending the sequencing primer binding to the sequencing primer binding sites.

8. The method of claim 7, wherein the pyrrolidone or amide has a structure selected from 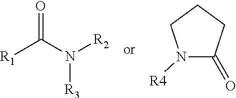
wherein R1 is H, methyl, propyl, or hydroxyethyl; R2 and R3 are independently of each other H or methyl; and R4 is H, propyl or isobutyl.

9. The method of claim 8, wherein the pyrrolidone is selected from 2-pyrrolidone, N-methyl-pyrrolidone, N-hydroxyethyl pyrrolidone, and the amide is selected from acetamide, N-methylacetamide, N,N-dimethyl acetamide, propionamide, isobutyramide.

10. The method of claim 9, wherein the sequencing is characterized by a performance characteristic equal to that of a method utilizing formamide-containing hybridization solution, wherein the characteristic is selected from read-on-target, deduplicated (deduped) depth, error rate, uniformity and GE recovery.

11. The method of claim 10, wherein the characteristic is read-on-target, and the read-on-target is about 70% or greater.

12. The method of claim 10, wherein the characteristic is deduplicated depth, and the deduplicated depth is 2500 or higher.

13. A kit for enriching for target nucleic acids to be sequenced by a single-molecule sequencing by synthesis, the kit comprising reagents for:
a. isolating nucleic acids in a sample solution;
b. conjugating the nucleic acids to adaptors, wherein the adaptors comprise universal primer binding sites and sequencing primer binding sites;
c. amplifying the adapted target nucleic acids with universal primers;
d. hybridization to single-stranded hybridization probes wherein the hybridization buffer comprises a solvent selected from dimethyl sulfoxide (DMSO), sulfolane, ethylene carbonate, pyrrolidone or a primary amide.

14. The kit of claim 13, wherein the pyrrolidone or amide has a structure selected from 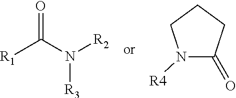
wherein R1 is H, methyl, propyl, or hydroxyethyl; R2 and R3 are independently of each other H or methyl; and R4 is H, propyl or isobutyl.

15. The kit of claim 13, wherein the pyrrolidone is selected from 2-pyrrolidone, N-methyl-pyrrolidone, N-hydroxyethyl pyrrolidone, and the amide is selected from acetamide, N-methylacetamide, N,N-dimethyl acetamide, propionamide, isobutyramide.

16. The kit of claim 13, further comprising one or more of the following: adaptors, universal amplification primers, a DNA ligase, a polynucleotide kinase, an exonuclease with a 5′-3′-activity, a sequencing DNA polymerase, and an amplification DNA polymerase.
